(19) United States
(12) Patent Application Publication
(10) Pub. No.: US 2012/0010290 A1
(43) Pub. Date: Jan. 12, 2012

(54) Methods of Treating an Overweight or Obese Subject

(76) Inventor: James E. Vath, Lynnfield, MA (US)

(21) Appl. No.: 13/133,055

(22) PCT Filed: Dec. 4, 2009
(86) PCT No.: PCT/US09/66811

§ 371 Date: Sep. 23, 2011

Related U.S. Application Data
(62) Division of application No. 61/119,872, filed on Dec. 4, 2008.
Application No. 61/119,872 is a division of application No. 61/275,688, filed on Aug. 3, 2009.
Application No. 61/275,688 is a division of application No. 61/260,194, filed on Nov. 11, 2009.

(51) Int. Cl. A61K 31/196; A61P 5/14; A61P 3/04

(52) U.S. Cl. 514/562

(57) ABSTRACT

The invention herein generally relates to methods of treating a subject having an overweight or obese condition, and overweight- or obesity-related conditions. In one embodiment, the invention herein provides a method of treating a subject having overweight or obese condition involving administering to the subject in need thereof, an amount of a pharmaceutical composition including a MetAP-2 inhibitory compound, or a salt, ester, or prodrug thereof, effective to result in weight loss in the subject.

### REFERENCE TO RELATED APPLICATIONS

This application claims priority to U.S. provisional applications U.S. Ser. No. 61/119,872 filed Dec. 4, 2008, U.S. Ser. No. 61/275,688 filed Aug. 3, 2009, and U.S. Ser. No. 61/260,194 filed Nov. 11, 2009, each application of which is hereby incorporated by reference.

### BACKGROUND

Obesity is a complex medical disorder of appetite regulation and metabolism resulting in excessive accumulation of adipose tissue mass. Typically defined as a body mass index (BMI) of 30 kg/m² or more, obesity is a world-wide public health concern that is associated with cardiovascular disease, diabetes, certain cancers, respiratory complications, osteoarthritis, gallbladder disease, decreased life expectancy, and work disability. The primary goals of obesity therapy are to reduce excess body weight, improve or prevent obesity-related morbidity and mortality, and maintain long-term weight loss.

Treatment modalities typically include lifestyle management, pharmacotherapy, and surgery. Treatment decisions are made based on severity of obesity, seriousness of associated medical conditions, patient risk status, and patient expectations. Notable improvements in cardiovascular risk and the incidence of diabetes have been observed with weight loss of 5-10% of body weight, supporting clinical guidelines for the treatment of obesity that recommend a target threshold of 10% reduction in body weight from baseline values.

However, while prescription anti-obesity medications are typically considered for selected subjects at increased medical risk because of their weight and for whom lifestyle modifications (diet restriction, physical activity, and behavior therapy) alone have failed to produce durable weight loss, approved drugs have had unsatisfactory efficacy ibr severely obese subjects, leading to only ˜3-5% reduction in body weight after a year of treatment.

Bariatric surgery may be considered as a weight loss intervention for subjects at or exceeding a BMI of 40 kg/m². Subjects with a BMI ≧35 kg/m² and an associated serious medical condition are also candidates for this treatment option. Unfortunately, postoperative complications commonly result from bariatric surgical procedures, including bleeding, embolism or thrombosis, wound complications, deep infections, pulmonary complications, and gastrointestinal obstruction; reoperation during the postoperative period is sometimes necessary to address these complications. Rates of reoperation or conversion surgery beyond the postoperative period depend on the type of bariatric procedure, and in one study ranged from 17% to 31%. Intestinal absorptive abnormalities, such as micronutrient deficiency and protein-calorie malnutrition, also are typically seen with bypass procedures, requiring lifelong nutrient supplementation. Major and serious adverse outcomes associated with bariatric surgery are common, observed in approximately 4 percent of procedures performed (including death in 0.3 to 2 percent of all patients receiving laparoscopic banding or bypass surgeries, respectively).

MetAP-2 encodes a protein that functions at least in part by enzymatically removing the amino terminal methionine residue from certain newly translated proteins such as glyceraldehyde-3-phosphate dehydrogenase (Warder et al. (2008) J Proteome Res 7:4807). Increased expression of the MetAP-2 gene has been historically associated with various forms of cancer. Molecules inhibiting the enzymatic activity of MetAP-2 have been identified and have been explored for their utility in the treatment of various tumor types (Wang et al. (2003) Cancer Res. 63:7861) and infectious diseases such as microsporidiosis, leishmaniasis, and malaria (Zhang et al. (2002) J. Biomed. Sci. 9:34). However, such MetAP-2 inhibitors may be useful as well for subjects with excess adiposity and conditions related to adiposity including type 2 diabetes, hepatic steatosis, and cardiovascular disease (via e.g. ameliorating insulin resistance, reducing hepatic lipid content, and reducing cardiac workload). Methods of treating obese subjects that are more effective than e.g. dieting alone are clearly needed.

### SUMMARY

The disclosure relates to methods for treating a subject having an overweight or obese condition or a condition related to being overweight obese with pharmaceutical compositions including MetAP-2 inhibitory compounds, or a pharmaceutically acceptable salt, ester, or prodrug thereof. In one aspect, the disclosure provides methods of treating a subject having an overweight or obese condition including, administering to the subject in need thereof, a therapeutically effective amount of a pharmaceutical composition including a compound of Formula I, II, III, IV, V, VI, VII, VIII, IX, X, XI, XII, XIII, XIV, XV, XVI, XVII, XVIII, XIX, XX, XIX, and/or another compound disclosed herein.

In certain embodiments, the pharmaceutical composition is administered non-parenterally, for example, orally, buccally, sublingually, transdermally, via inhalation, or rectally.

In one embodiment, administration results in decreased body fat and a substantial maintenance of muscle mass in the subject. In another embodiment, upon administration, fat oxidation is enhanced as compared to a subject on a restricted food intake diet alone. In another embodiment, substantially no loss of new blood vessels in fat deposits occur as compared to a subject being treated for obesity using an energy restricted diet alone.

In one aspect, a disclosed method relates to controlling or preventing hepatic steatosis in an obese subject being treated for obesity, comprising administering a therapeutically effective amount of a pharmaceutical composition including a compound disclosed herein. Also provided herein is a method relating to improving liver function in an obese subject, including administering a therapeutically effective amount of a pharmaceutical composition including a compound described herein to the subject.

In another aspect, a disclosed method relates to improving exercise capacity in a subject in need thereof comprising administering a therapeutically effective amount of a pharmaceutical composition including a compound described herein to the subject.

A method relating to reducing weight of a subject in a subject in need thereof is also contemplated herein, including administering a therapeutically effective amount of a pharmaceutical composition including a compound described herein to the subject. For example, the metabolic rate of the subject may not substantially reduced as compared to the metabolic rate of a subject on an energy restricted diet alone. In another aspect, a disclosed method relates to restoring normal metabolic action in an obese subject in need thereof, including administering a therapeutically effective amount of a pharmaceutical composition including a compound disclosed herein to the subject.

Also provided herein is a method relating to decreasing body fat in an overweight or obese subject in need thereof comprising administering a therapeutically effective amount of a pharmaceutical composition comprising a compound disclosed herein to the subject resulting in body fat reduction and substantial maintenance of muscle mass during the body fat reduction. In one embodiment, the subject retains substantially more muscle mass as compared to body fat reduction in a subject using an energy restricted diet alone.

In another aspect, a disclosed method relates to activating brown fat function and/or increasing brown fat tissue mass in a subject in need thereof, including administering a therapeutically effective amount of a pharmaceutical composition including a compound disclosed herein to the subject.

In another aspect, a disclosed method relates to restoring and/or maintaining thyroid hormone concentrations in an obese subject, including administering a therapeutically effective amount of a pharmaceutical composition including a compound described herein to the subject.

In one embodiment, the therapeutically effective amount does not substantially modulate or suppress angiogenesis. In another embodiment, the subject has a lower systemic exposure to said compound as compared to a subject parenterally administered the same amount of the compound.

In an exemplary embodiment, disclosed methods such as a method relating to treating a subject having an overweight or obese condition, includes administering to the subject a therapeutically effective amount of a pharmaceutical composition comprising the compound

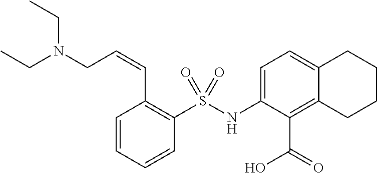

### DETAILED DESCRIPTION

### Overview

The disclosure provides methods of reducing adipose tissue in an overweight subject using MetAP-2 inhibitory compounds, such as those disclosed herein. A MetAP-2 inhibitory compound includes a moiety able to inhibit the activity of methionine aminopeptidase 2 (MetAP-2), e.g., the ability of MetAP-2 to cleave the N-terminal methionine residue of newly synthesized proteins to produce the active form of the protein. Exemplary MetAP-2 inhibitory compounds are fumagillin derived structures. Further examples of MetAP-2 inhibitory compounds are shown in the following U.S. patents, U.S. patent applications, PCI international patent applications, and published papers: BaMaung et al. (U.S. Pat. No. 7,030,262), Craig et al. (WO 1999/057097), Craig et al. (U.S. Pat. No. 6,887,863), Henkin et al. (WO 2002/083065), Henkin et al. (WO 2002/026782), Comess et al. (WO 2004/033419), Comess et al. (US 2004/0068012), Craig et al. (U.S. Pat. No. 6,242,494), Craig et al. (US 2002/0002152), Comess et al. (US 2004/0157836), Comess et. al. (US 2004/0167128), Sheppard et al. (2004, Bioorganic & Medicinal Chemistry Letters 14:865-868), Wang et al. (2003, Cancer Research 63:7861-7869), Kawai et al. (2006, Bioorganic & Medicinal Chemistry Letters 16:3574-3577), and Wang et al. (2007, Bioorganic & Medicinal Chemistry Letters 17:2817-2822).

Obesity and being overweight refer to an excess of fat in proportion to lean both mass. Excess fat accumulation is associated with increase in size (hypertrophy) as well as number (hyperplasia) of adipose tissue cells. Obesity is variously measured in terms of absolute weight, weight:height ratio, distribution of subcutaneous fat, and societal and esthetic norms. A common measure of body fat is Body Mass Index (BMI). The BMI refers to the ratio of body weight (expressed in kilograms) to the square of height (expressed in meters).

In accordance with the U.S. Centers for Disease Control and Prevention (CDC), an overweight adult has a BMI of 25 kg/m² to 29.9 kg/m², and an obese adult has a BMI of 30 kg/m² or greater. BMI of 40 kg/m² or greater is indicative of morbid obesity or extreme obesity. For children, the definitions of overweight and obese take into account age and gender effects on body fat.

BMI does not account for the fact that excess adipose can occur selectively in different parts of the body, and development of adipose tissue can be more dangerous to health in some parts of the body rather than in other parts of the body. For example, “central obesity”, typically associated with an “apple-shaped” body, results from excess adiposity especially in the abdominal region, including belly fat and visceral fat, and carries higher risk of co-morbidity than “peripheral obesity”, which is typically associated with a “pear-shaped” body resulting from excess adiposity especially on the hips. Measurement of waist/hip circumference ratio (WHR) can be used as an indicator of central obesity. A minimum WHR indicative of central obesity has been variously set, and a centrally obese adult typically has a WHR of about 0.85 or greater if female and about 0.9 or greater if male.

Methods of determining whether a subject is overweight or obese that account for the ratio of excess adipose tissue to lean body mass involve obtaining a body composition of the subject. Body composition can be obtained by measuring the thickness of subcutaneous fat in multiple places on the body, such as the abdominal area, the subscapular region, arms, buttocks and thighs. These measurements are then used to estimate total body fat with a margin of error of approximately four percentage points. Another method is bioelectrical impedance analysis (BIA), which uses the resistance of electrical flow through the body to estimate body fat. Another method is using a large tank of water to measure body buoyancy. Increased body fat will result in greater buoyancy, while greater muscle mass will result in a tendency to sink. Another method is fan-beam dual energy X-ray absorptiometry (DEXA). DEXA allows body composition, particularly total body fat and/or regional fat mass, to be determined non-invasively.

Without being limited by any particular theory or mechanism of action, it is believed that fin oxidation and lipolysis are stimulated through treatment with inhibitors of MetAP2 that enhance the level and function of thioredoxin and/or over-rides the inhibitory effects of hyperinsulinemia related at least in part to insulin-stimulation and/or over-rides the inhibitory effects of high fat diet induced NADPH oxidase activity. A coordinated action can be induced which leads to a physiological reduction in body adiposity through increased loss of fat tissue-associated triglyceride, enhanced local generation of 3,5,3′-triiodothyronine active thyroid hormone with corresponding enhanced activity of brown adipose tissue and its sensitivity to physiological stimuli, increased metabolism of free fatty acids by the liver with increased ketone body formation, and reduced food intake. These effects are evident at doses of a MetAP2 inhibitor that do not substantially modulate angiogenesis.

In obese and/or hyperinsulinemic subjects, liver PKA function may be suppressed secondary to elevated NADPH oxidase expression. Ketone body production and utilization are typically suppressed in an obese subject, potentially reducing hepatic satiety signals and increasing food consumption. However, administration of a MetAP2 inhibitor, without being limited by an theory, leads to inhibition of thioredoxin amino-terminal methionine processing and increases steady-state thioredoxin levels, reactivating protein kinase A (PKA) function, reactivating adipose tissue lipase activity and/or stimulating production and/or activity of the rate-limiting enzyme of beta-hydroxybutyrate production (3-hydroxymethyl glutaryl CoA synthase), leading to elevated ketone body production.

The coordinated and physiologic induction of anti-obesity activities mediated by the methods disclosed herein may lead to a healthy reduction in tissue levels of triglyceride, diacylglycerol, and other fat-related mediators and oxidants, and can result in a new steady state situation that favors lean body composition and increased whole body energy metabolism. Without being bound by any theory, it is believed that the mechanistic cascade activated by MetAP2 inhibitors leads to fat tissue being converted to ketone bodies and burned as fuel, unlike existing therapies (including e.g., calorie or energy restricted diets) that target central control of food intake and that may carry adverse side effects (e.g. adverse neurological side effects). Further, therapeutically effective doses contemplated herein will not typically induce any anti-angiogenic action.

An effective therapy for treating an overweight or obese subject reduces adipose tissue without resulting in deleterious side effects, for example, wasting. Wasting is characterized by degradation and loss of a substantial amount of lean body mass (muscle tissue, bones, and organs) in addition to adipose tissue. In particular, lean body mass refers to structural and functional elements in cells, body water, muscle, bones, and other body organs such as the heart, liver, and kidneys. Although weight loss may involve loss of fat along with slight loss of muscle or fluid, weight loss for the purposes of maintaining health should aim to lose fat while conserving lean body mass. Wasting involves uncontrollable weight loss.

Treatment-induced wasting may occur as a side-effect of some drugs. High-dose sulphonamides, anti-mycobacterial agents, and other medications have been associated with anorexia and subsequent wasting. Substantial loss of lean body mass can lead to various diseases. Schaafsma (Current Topics in Nutraceutical Research (2006) ISSN 1540-7535 4(2):113-121). Health problems associated with loss of lean body mass include difficulty fighting off infection, osteoporosis, decreased muscle strength, trouble regulating body temperature, and even increased risk of death.

### MetAP-2 Compounds

Compounds for use with the methods disclosed herein include compounds that result in the desirable effect of a reduction in adipose tissue but without resulting in deleterious side effects, for example, wasting. In certain embodiments, the compounds are compounds of Formula I:

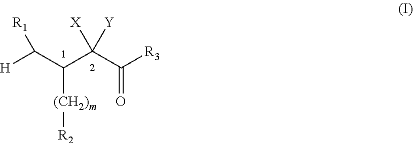

or a pharmaceutically acceptable salt, ester, or prodrug thereof, wherein: the small numerals denote chiral centers in the compound; m is 1-3; R₁ is selected from the group consisting of hydrogen, alkyl, carboxaldehyde, alkanoyl, where the alkanoyl can be optionally substituted with hydroxyl, and —(CH₂)ₙCO₂R₄ where n is 0-6, and R₄ is selected from the group consisting of hydrogen, alkyl, cycloalkyl, (cycloalkyl)alkyl, aryl, and arylalkyl; where cycloalkyl and (cycloalkyl)alkyl can be optionally substituted with 1, 2, or 3 substituents independently selected from the group consisting of alkyl, alkoxy, and aryl; and where aryl and arylalkyl can be optionally substituted with 1, 2, or 3 substituents independently selected from the group consisting of alkyl, alkanoyl, alkoxy, —CO₂R₄ where R_{4′} is selected from the group consisting of, hydrogen, alkyl, cycloalkyl, (cycloalkyl)alkyl, aryl, and arylalkyl.

R₁ can further include alkanoyloxy, carboxaldehyde, cycloalkyl, cycloalkenyl, halo, nitro, perfluoroalkyl, perfluoroalkoxy, arylsulfonylalkyl, aryloylalkyloxycarbonylalkyl, —NR₆R_{6′}, where R₆ and R_{6′} are independently selected from the group consisting of hydrogen, alkyl optionally substituted with alkoxy, aryl, arylalkyl, and a nitrogen-protecting group, SO₂NR₆R_{6′}, where R₆ and R_{6′} are defined above, and —C(O)NR₆R_{6′}, where R₆ and R_{6′} are defined above.

R₂ is selected from the group consisting of alkyl, cycloalkyl, (cycloalkyl)alkyl, —C(H)(SR₁₅)(SR_{15′}), where R₁₅ and R_{15′} are alkyl, or R₁₅ and R_{15′}, together with the sulfurs to which they are attached, are a 1,3-dithiolane ring or a 1,3-dithiane ring, aryl, arylalkyl.

R₂ can further include —SR₅, where R₅ is selected from the group consisting of alkyl, cycloalkyl, (cycloalkyl)alkyl, and benzyl; where the benzyl can be optionally substituted with 1, 2, or 3 substituents independently selected from the group consisting of alkyl, alkanoyl, alkoxy.

R₂ can further include —CO₂R₄, where R₄ is defined above, alkanoyloxy, carboxaldehyde, cycloalkyl, cycloalkenyl, halo, nitro, perfluoroalkyl, perfluoroalkoxy, NR₆R_{6′}, where R₆ and R_{6′} are defined above, —SO₂NR₆R_{6′}, where R₆ and R_{6′} are defined above, and —C(O)NR₆R_{6′}, where R₆ and R_{6′} are defined above.

R₃ is selected from the group consisting of an aminoacyl group optionally capped with a carboxyl protecting group, —N(R₆)(CH₂)ₚR₇, where p is 0-6, R₆ is defined above, and R₇ is selected from the group consisting of hydrogen, alkyl, where the alkyl can be optionally substituted with 1, 2, 3, or 4 substituents independently selected from the group consisting of oxo, thioxo, alkoxy, —CO₂R₄, where R₄ is defined above, alkanoyloxy, carboxaldehyde, cycloalkyl, cycloalkenyl, halo, nitro, perfluoroalkyl, perfluoroalkoxy, —NR₆R_{6′}, where R₆ and R_{6′} are defined above, —SO₂NR₆R_{6′}, where R₆ and R_{6′} are defined above, —C(O)NR₆R_{6′}, where R₆ and R_{6′} are defined above, aryl, hydroxy, and heterocycle.

R₃ can further include cycloalkyl, where the aryl can be optionally substituted with 1, 2, or 3 substituents independently selected from the group consisting of alkyl, halo, oxo, and aryl.

R₃ can further include aryl, where the aryl can be optionally substituted with 1, 2, or 3 substituents independently selected from the group consisting of alkyl, alkanoyl, alkoxy, —CO₂R₄, Where R₄ is defined above, alkanoyloxy, carboxaldehyde, cycloalkyl, cycloalkenyl, halo, nitro, perfluoroalkyl, perfluoroalkoxy, —NR₆R_{6′}, where R₆ and R_{6′} are defined above, —SO₂NR₆R_{6′}, where R₆ and R_{6′} are defined above, —C(O)NR₆R_{6′}, where R₆ and R_{6′} are defined above, aryloxy, arylalkoxy, aryl, hydroxy, and heterocycle.

R₃ can further include where R₄ is defined above, —CONR₆R₈, where R₆ is defined above, and R₈ is selected from the group consisting of hydrogen alkyl, aryl, and heterocycle, where alkyl, aryl, and heterocycle can be optionally substituted with one, two, or three groups independently selected from the group consisting of alkyl, alkanoyl, alkoxy, —CO₂R₄, where R₄ is defined above, alkanoyloxy, carboxaldehyde, cycloalkyl, cycloalkenyl, halo, nitro, perfluoroalkyl, perfluoroalkoxy —NR₆R_{6′}, where R₆ and R_{6′} are defined above, —SO₂NR₆R_{6′}, where R₆ and R_{6′} are defined above, —C(O)NR₆R_{6′}, where R₆ and R_{6′} defined above, aryloxy, arylalkoxy, aryl, hydroxy, and heterocycle.

R₃ can further include heterocycle, where the heterocycle can be optionally substituted with one, two, or three groups independently selected from the group consisting of alkyl, alkanoyl, alkoxy, —CO₂R₄, where R₄ is defined above, alkanoyloxy, carboxaldehyde, cycloalkyl, cycloalkenyl, halo, nitro, perfluoroalkyl, perfluoroalkoxy, —NR₆R_{6′}, where R₆ and R_{6′} are defined above, —SO₂NR₆R_{6′}, where R₆ and R_{6′} are defined above, —C(O)NR₆R_{6′}, where R₆ and R_{6′} are defined above, aryloxy, arylalkoxy, aryl, hydroxy, and heterocycle.

R₃ can further include —NR₆R₈, where R₆ and R₈ are defined above, and —N(R₆)SO₂R₁₂, where R₆ is defined previously, and R₁₂ is selected from the group consisting of alkyl, aryl, arylalkyl, heterocycle, and (heterocycle)alkyl, where aryl, arylalkyl, heterocycle, and (heterocycle)alkyl can be optionally substituted with 1, 2, or 3 groups independently selected from the group consisting of alkyl, alkanoyl, alkoxy, —CO₂R₄, where R₄ is defined above, alkanoyloxy, carboxaldehyde, cycloalkyl, cycloalkenyl, halo, nitro, perfluoroalkyl, perfluoroalkoxy, —NR₆R_{6′}, where R₆ and R_{6′} are defined above, —SO₂NR₆R_{6′}, where R₆ and R_{6′} are defined above, —C(O)NR₆R_{6′}, where R₆ and R_{6′} are defined above, aryloxy, arylalkoxy, aryl, hydroxy, and heterocycle.

R₃ can further include —O(CH₂)ₚR₇ where p and R₇ are defined above, and —NR₂₀R₂₁, where R₂₀ and R₂₁, together with the nitrogen atom to which they are attached, are a 3- to 7-membered ring optionally containing therein 1 or 2 double bonds and optionally containing therein a moiety selected from the group consisting of oxygen, nitrogen and —S(O)ₓ—, wherein x is 0-2, where the ring formed by R₂₀ and R₂₁ can be optionally substituted with 1, 2, or 3 groups independently selected from the group consisting of alkyl, alkanoyl, alkoxy, —CO₂R₄, where R₄ is defined above, alkanoyloxy, carboxaldehyde, cycloalkyl, cycloalkenyl, halo, nitro, perfluoroalkyl, perfluoroalkoxy, —NR₆R_{6′}, where R₆ and R_{6′} are defined above. SO₂NR₆R_{6′}, where R₆ and R_{6′} are defined above, —C(O)NR₆R_{6′}, where R₆ and R_{6′} are defined Above, aryloxy, arylalkoxy, aryl, hydroxy, and heterocycle.

X is hydroxyl or sulfhydryl, Y is hydrogen; or X and Y, taken together with the carbon atom to which they are attached, form a carbonyl or thiocarbonyl.

Further definitions and examples of substituents for each moiety in Formula I are shown in Craig et al. (WO 1999/057097) and Craig et al. (U.S. Pat. No. 6,242,494). Further embodiments and examples of the compounds of Formula I are shown in Craig et al. (WO 1999/057097) and Craig et al. (U.S. Pat. No. 6,242,494). Methods of making compounds of Formula I are shown in Craig et al. (WO 1999/057097) and Craig et al. (U.S. Pat. No. 6,242,494).

In other embodiments, the compounds are compounds of Formula II:

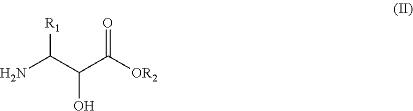

or a pharmaceutically acceptable salt, ester, or prodrug thereof, wherein: R₁ is selected from alkyl, alkylsulfanylalkyl, aryl, arylalkyl, cycloalkyl, (cycloalkyl)alkyl, (heterocycle)alkyl, and hydroxyalkyl; and R₂ is selected from the group consisting of hydrogen, alkenyl, alkyl, alkylcarbonyloxyalkyl, alkylcarbonylalkyl, aryl, arylalkyl, cycloalkyl, (cycloalkyl)alkyl, heterocycle, and (heterocycle)alkyl.

Further definitions and examples of substituents for each moiety in Formula II are shown in BaMaung et al. (U.S. Pat. No. 7,030,262). Further embodiments and examples of the compounds of Formula II are shown in BaMaung et al. (U.S. Pat. No. 7,030,262). Methods of making compounds of Formula II are shown in BaMaung et al. (U.S. Pat. No. 7,030,262).

In other embodiments, the compounds are compounds of Formula III:

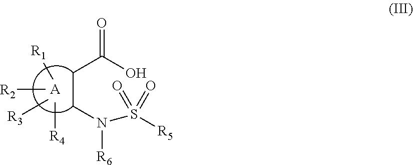

or a pharmaceutically acceptable salt, ester, or prodrug thereof, wherein: A is a five- or six-membered aromatic or non-aromatic ring containing from zero to three atoms selected from the group consisting of nitrogen, oxygen, and sulfur; wherein the five- or six-membered ring is optionally fused to a second five-, six-, or seven-membered aromatic or non-aromatic ring containing from zero to three atoms selected from the group consisting of nitrogen, oxygen, and sulfur.

R₁, R₂ and R₃ are independently selected from the group consisting of hydrogen, alkenyl, alkoxy, alkoxyalkyl, alkoxycarbonyl, alkoxycarbonylalkyl, alkyl, alkylcarbonyloxy, alkylidene, alkylsulfanyl, alkylsulfanylalkyl, alkylsulfonyl, alkylsulfonylalkyl, amino, aminoalkyl, aminoalkenyl, aminoalkoxy, aminocarbonylalkenyl, aryl, carboxyalkenyl, carboxyalkyl, cyano, cycloalkyl, (cycloalkyl)alkyl, halo, haloalkoxy, haloalkyl, (heterocycle) alkyl, hydroxy, hydroxyalkyl, nitro.

R₄ is selected from the group consisting of hydrogen, alkenyl, alkoxy, alkoxyalkyl, alkoxycarbonyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c5}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, R_{c4}R_{f4}Nalkyl(R_{c4})Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4}R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulthnyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and arylalkyl. In some embodiments, when A is phenyl, at least one of R₁, R₂, R₃ and R₄ is other than hydrogen, Cl alkyl or halo.

Further definitions and examples of substituents for each moiety in Formula III are shown in Comess et al. (WO 2004/033419), Comess et al. (US 2004/0157836), and Comess et al. (US 2004/0167128). Further embodiments and examples of the compounds of Formula III are shown in Comess et al. (WO 2004/033419), Comess et al. (US 2004/0157836), and Comess et al. (US 2004/0167128). Methods of making compounds of Formula III are shown in Comess et al. (WO 2004/033419), Comess et al. (US 2004/0157836), and Comess et al. (US 2004/0167128).

In other embodiments, the compounds are compounds of Formula IV:

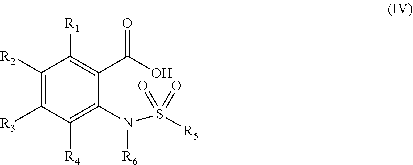

or a pharmaceutically acceptable salt, ester, or prodrug thereof wherein: R₁, R₂ and R₃ are independently selected from the group consisting of hydrogen, alkenyl, alkoxy, alkoxyalkyl, alkoxycarbonyl, alkoxycarbonylalkyl, alkyl, alkylcarbonyloxy, alkylidene, alkylsulfanyl, alkylsulfanylalkyl, alkylsulfonyl, alkylsulfonylalkyl, amino, aminoalkyl, aminoalkenyl, aminoalkoxy, aminocarbonylalkenyl, aryl, carboxyalkenyl, carboxyalkyl, cyano, cycloalkyl, (cycloalkyl)alkyl, halo, haloalkoxy, haloalkyl, (heterocycle) alkyl, hydroxy, hydroxyalkyl, nitro; or R₁ and R₂ together with the carbon atoms to which they are attached, form a five-, six-, or seven-membered saturated or unsaturated carbocyclic ring which can be optionally substituted with 1 or 2 substituents independently selected from the group consisting of alkoxy, alkyl, amino, halo, and haloalkyl; or R₂ and R₃ together with the carbon atoms to which they are attached, form olive-, six, or seven-membered saturated or unsaturated carbocyclic ring which can be optionally substituted with 1 or 2 substituents independently selected from the group consisting of alkoxy, alkyl, amino, halo, and haloalkyl.

R₄ is selected from the group consisting of hydrogen, alkenyl, alkoxy, alkoxyalkyl, alkoxycarbonyl, alkyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R_{c4})Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, cyano, halo, haloalkoxy, haloalkyl, and nitro; and wherein R_{c4} R_{d4} Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and arylalkyl; and provided that at least one of R₁, R₂, R₃ and R₄ is other than hydrogen, Cl alkyl or halo.

Further definitions and examples of substituents for each moiety in Formula IV are shown in Comess et al. (WO 2004/033419), Comess et al. (US 2004/0157836), and Comess et al. (US 2004/0167128). Further embodiments and examples of the compounds of Formula IV are shown in Comess et al. (WO 2004/033419), Comess et al. (US 2004/0157836), and Comess et al. (US 2004/0167128). Methods of making compounds of Formula IV are shown in Comess et al. (WO 2004/033419), Comess et al. (US 2004/0157836), and Comess et al. (US 2004/0167128).

In other embodiments, the compounds are compounds of Formula V:

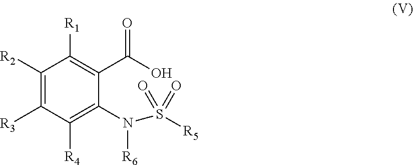

or a pharmaceutically acceptable salt, ester, or prodrug thereof, wherein: R₁ is selected from the group consisting of hydrogen, C₁-C₄ alkyl, C₂-C₄ alkenyl, C₁-C₄ alkoxy, halo, haloalkyl, haloalkoxy, RₐR_{b}N— and RₐR_{b}Nalkoxy, wherein Rₐ and R_{b} are each independently selected from the group consisting of hydrogen and alkyl.

R₂ is selected from the group consisting of alkoxy, alkoxyalkyl, C₁-C₁₀ alkyl, alkylsulfanyl, alkylsulfanylalkyl, alkylsulfonyl, alkylsulfonylalkyl, amino, aminoalkyl, cycloalkyl, (cycloalkyl)alkyl, halo, haloalkoxy, and haloalkyl. R₃ is selected from the group consisting of hydrogen, alkyl and halogen.

R₄ is selected from the group consisting of hydrogen, alkenyl, alkoxy, alkoxyalkyl, alkoxycarbonyl, alkyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R_{c4})Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4} R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{cs})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and arylalkyl.

Further definitions and examples of substituents for each moiety in Formula V are shown in Comess et al. (WO 2004/033419), Comess et al. (US 2004/0157836), and Comess et al. (US 2004/0167128). Further embodiments and examples of the compounds of Formula V are shown in Comess et al. (WO 2004/033419), Comess et al. (US 2004/0157836), and Comess et al. (US 2004/0167128). Methods of making compounds of Formula V are shown in Comess et al. (WO 2004/033419), Comess et al. (US 2004/0157836), and Comess et al. (US 2004/0167128).

In other embodiments, the compounds are compounds of Formula VI:

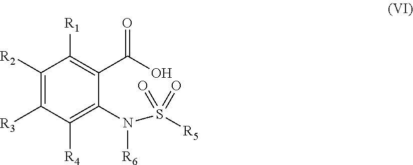

or a pharmaceutically acceptable salt, ester, or prodrug thereof, wherein: R₁ and R₂, together with the carbon atoms to which they are attached, form a five-, six, or seven-membered saturated or unsaturated carbocyclic ring which can be optionally substituted with one or two substituents independently selected from the group consisting of alkoxy, alkyl, amino, halo, and haloalkyl. R₃ is selected from the group consisting of hydrogen, alkyl and halogen.

R₄ is selected from the group consisting of hydrogen, alkenyl, alkoxy, alkoxyalkyl, alkoxycarbonyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R_{c4})Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4} R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(Rₑ₅)Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and aralkyl.

Further definitions and examples of substituents for each moiety in Formula VI are shown in Comess et al. (WO 2004/033419), Comess et al. (US 2004/0157836), and Comess et al. (US 2004/0167128). Further embodiments and examples of the compounds of Formula VI are shown in Comess et al. (WO 2004/033419), Comess et al. (US 2004/0157836), and Comess et al. (US 2004/0167128). Methods of making compounds of Formula VI are shown in Comess et al. (WO 2004/033419), Comess et al. (US 2004/0157836), and Comess et al. (US 2004/0167128).

In other embodiments, the compounds are compounds of Formula VII:

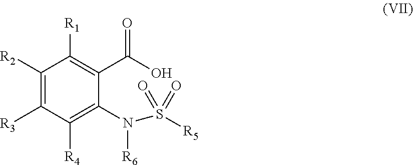

or a pharmaceutically acceptable salt, ester, or prodrug thereof, wherein: R₁ and R₂, together with the carbon atoms to which they are attached, form a six membered monounsaturated carbocyclic ring which can be optionally substituted with one or two substituents independently selected from the group consisting of alkoxy, alkyl, amino, halo, and haloalkyl. R₃ is selected from the group consisting of hydrogen, alkyl and halogen.

R₄ is selected from the group consisting of hydrogen, alkenyl, alkoxy, alkoxyalkyl, alkoxycarbonyl, alkyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R_{c4})Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(R_{c4})Ncarbonyl(Rₑ₄)N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4} R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of Rₑ₄ and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and arylalkyl.

Further definitions and examples of substituents for each moiety in Formula VII are shown in Comess et al. (WO 2004/033419), Comess et al. (US 2004/0157836), and Comess et al. (US 2004/0167128). Further embodiments and examples of the compounds of Formula VII are shown in Comess et al. (WO 2004/033419), Comess et al. (US 2004/0157836), and Comess et al. (US 2004/0167128). Methods of making compounds of Formula VII are shown in Comess et al. (WO 2004/033419), Comess et al. (US 2004/0157836), and Comess et al. (US 2004/0167128).

In other embodiments, the compounds are compounds of Formula VIII:

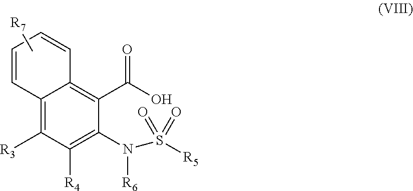

or a pharmaceutically acceptable salt, ester, or prodrug thereof, wherein: R₃ is selected from the group consisting of hydrogen, alkyl and halogen.

R₄ is selected from the group consisting of hydrogen, alkenyl, alkoxy, alkoxyalkyl, alkoxycarbonyl, alkyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R₄)Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4} R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, aryl alkenyl, aryl alkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and arylalkyl. R₇ is selected from the group consisting of hydrogen, C₁-C₃ alkyl, C2-C₃ alkenyl, C₂-C₃ alkoxy, halo, haloalkyl, haloalkoxy, RₐR_{b}N— and RₐR_{b}Nalkoxy, wherein Rₐ and R_{b} are each independently selected from the group consisting of hydrogen and alkyl.

Further definitions and examples of substituents for each moiety in Formula VIII are shown in Comess et al. (WO 2004/033419), Comess et al. (US 2004/0157836), and Comess et al. (US 2004/0167128). Further embodiments and examples of the compounds of Formula VIII are shown in Comess et al. (WO 2004/033419), Comess et al. (US 2004/0157836), and Comess et al. (US 2004/0167128). Methods of making compounds of Formula VIII are shown in Comess et al. (WO 2004/033419), Comess et al. (US 2004/0157836), and Comess et al. (US 2004/0167128).

In other embodiments, the compounds are compounds of Formula IX:

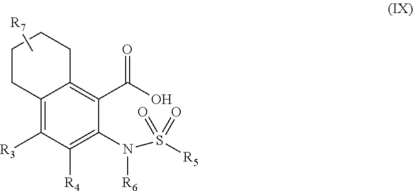

or a pharmaceutically acceptable salt, ester, or prodrug thereof, wherein: R₃ is selected from the group consisting of hydrogen, alkyl and halogen.

R₄ is selected from the group consisting of hydrogen, alkenyl, alkoxy, alkoxyalkyl, alkoxycarbonyl, alkyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R_{c4})Ncarbonylalkenyl, R_{c4}R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4} R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(Rₑ₅)N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocycloalkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and arylalkyl. R₇ is selected from the group consisting of hydrogen, C₁-C₃ alkyl, C2-C₃ alkenyl, C₂-C₃ alkoxy, halo, haloalkyl, haloalkoxy, RₐR_{b}N— and RₐR_{b}Nalkoxy, wherein Rₐ and R_{b} are each independently selected from the group consisting of hydrogen and alkyl.

Further definitions and examples of substituents for each moiety in Formula IX are shown in Comess et al. (WO 2004/033419), Comess et al. (US 2004/0157836), and Comess et al. (US 2004/0167128). Further embodiments and examples of the compounds of Formula IX are shown in Comess et al. (WO 2004/033419), Comess et al. (US 2004/0157836), and Comess et al. (US 2004/0167128). Methods of making compounds of Formula IX are shown in Comess et al. (WO 2004/033419), Comess et al. (US 2004/0157836), and Comess et al. (US 2004/0167128).

In other embodiments, the compounds are compounds of Formula X:

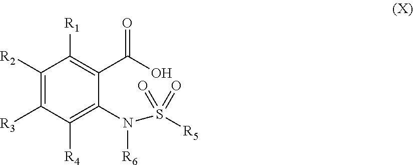

or a pharmaceutically acceptable salt, ester, or prodrug thereof, wherein: R₁ is selected from the group consisting of hydrogen, C₁-C₄ alkyl, C₂-C₄ alkenyl, C₂-C₄ alkoxy, halo, haloalkyl, haloalkoxy, RₐR_{b}N— and RₐR_{b}Nalkoxy, wherein Rₐ and R_{b} are each independently selected from the group consisting of hydrogen and alkyl.

R₂ and R₃, together with the carbon atoms to which they are attached, form a five-, six, or seven-membered saturated or unsaturated carbocyclic ring which can be optionally substituted with one or two substituents independently selected from the group consisting of alkoxy, alkyl, amino, halo, and haloalkyl.

R₄ is selected from the group consisting of hydrogen, alkenyl, alkoxy, alkoxyalkyl, alkoxycarbonyl, alkyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R_{c4})Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4} R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle.

R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein Rₑ₅, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl.

R₆ is selected from the group consisting of hydrogen, alkyl, alkylsulfanylalkyl, aryl, and arylalkyl.

Further definitions and examples of substituents for each moiety in Formula X are shown in Comess et al. (WO 2004/033419), Comess et al. (US 2004/0157836), and Comess et al. (US 2004/0167128). Further embodiments and examples of the compounds of Formula X are shown in Comess et al. (WO 2004/033419), Comess et al. (US 2004/0157836), and Comess et al. (US 2004/0167128). Methods of making compounds of Formula X are shown in Comess et al. (WI) 2004/033419), Comess et al. (US 2004/0157836), and Comess et al. (US 2004/0167128).

In other embodiments, the compounds are compounds of Formula XI:

N-A-Sar-Gly-AA³-AA⁴-AA⁵-AA⁶-AA⁷-Arg-Pro-AA¹⁰

or a pharmaceutically acceptable salt, ester, or prodrug thereof, wherein: AA³ is selected from the group consisting of glutaminyl, phenylalanyl, valyl, and asparaginyl; AA⁴ is selected from the group consisting of D-isoleucyl, isoleucyl, D-leucyl, and D-alloisoleucyl; AA is selected from the group consisting of seryl, methionyl, allothreonyl, threonyl, and tyrosyl; AA⁶ is selected from the group consisting of norvalyl, seryl, tryptophyl, glutaminyl, and prolyl; AA⁷ is selected from the group consisting of isoleucyl. D-isoleucyl, lysyl (acetyl), and prolyl; and AA¹⁰ is selected from the group consisting of D-alanylamide, ethylamide, and isopropylamide; with the proviso that one of AA⁴ and AA⁷ is a D-amino acid.

Further exemplary amino acids, and exemplary peptides of Formula XI are shown in Henkin et al. (WO 2002/083065). Methods of making peptides of Formula XI are shown in Henkin et al. (WO 2002/083065).

In other embodiments, the compounds are compounds of Formula XII:

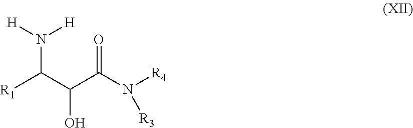

or a pharmaceutically acceptable salt, ester, or prodrug thereof, wherein: R₁ is selected from the group consisting of alkyl, aryl, arylalkyl, cycloalkyl, (cycloalkyl)alkyl, (heterocycle)alkyl, and R₅S-(alkylene)-; wherein each group is drawn with its right-hand end being the end that is attached to the parent molecular moiety; R₃ is selected from the group consisting of hydrogen, alkyl, and arylalkyl; R₄ is selected from the group consisting of —NR₆R₇, and —OR₈; wherein each group is drawn with its left-hand end being the end that is attached to the parent molecular moiety; R₅ is selected from the group consisting of alkyl, aryl, arylalkyl, cycloalkyl, and (cycloalkyl)alkyl; R₆ and R₇ are independently selected from the group consisting of hydrogen, alkanoyl, alkenyl, alkenyloxyalkyl, alkoxyalkyl, alkoxycarbonylalkyl, alkyl, alkylsulfanylalkyl, aryl, arylalkanoyl, arylalkoxyalkyl, arylalkoxycarbonyl, arylalkyl, aryloxyalkyl, (aryl)oyl, arylsulfonyl, carboxyalkyl, cycloalkyl, (cycloalkyl)alkyl, (cycloalkyl)alkanoyl, (cycloalkyl)oyl, haloalkanoyl, haloalkyl, heterocycle, (heterocycle)alkanoyl, (heterocycle)oyl, hydroxyalkyl, a nitrogen protecting group, and —C(O)NR₉R₁₀; or R₆ and R₇ together are arylalkylidene; or R₆ and R₇, together with the nitrogen atom to which they are attached, form a heterocycle; R₈ is selected from the group consisting of hydrogen, alkanoylalkyl, alkenyl, alkoxycarbonylalkyl, alkyl, amidoalkyl, aryl, arylalkyl, arylalkoxycarbonylalkyl, (aryl)oylalkyl, carboxyalkyl, and (cycloalkyl)alkyl; and R₉ and R₁₀ are independently selected from the group consisting of hydrogen, alkyl and aryl.

Further definitions and examples of substituents for each moiety in Formula XII are shown in Craig et al. (U.S. Pat. No. 6,887,863) and Craig et al. (US 2002/0002152). Further embodiments and examples of the compounds of Formula XII are shown in Craig et al. (U.S. Pat. No. 6,887,863) and Craig et al. (US 2002/0002152). Methods of making compounds of Formula XII are shown in Craig et al. (U.S. Pat. No. 6,887,863) and Craig et al. (US 2002/0002152).

In other embodiments, the compounds are compounds of Formula XIII:

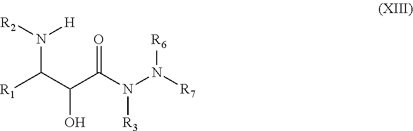

or a pharmaceutically acceptable salt, ester, or prodrug thereof, wherein: R₁ is selected from the group consisting of alkyl, aryl, arylalkyl, cycloalkyl, (cycloalkyl)alkyl, (heterocycle)alkyl, and R₅S-(alkylene)-; wherein each group is drawn with its right-hand end being the end that is attached to the parent molecular moiety; R₃ is selected from the group consisting of hydrogen, alkyl, and arylalkyl; R₅ is selected from the group consisting of alkyl, aryl, arylalkyl, cycloalkyl, and (cycloalkyl)alkyl; R₆ and R₇ are independently selected from the group consisting of hydrogen, alkanoyl, alkenyl, alkenyloxyalkyl, alkoxyalkyl, alkoxycarbonylalkyl, alkylsulfanylalkyl, aryl, arylalkanoyl, arylalkoxyalkyl, arylalkoxycarbonyl, arylalkyl, aryloxyalkyl, (aryl)oyl, arylsulfonyl, carboxyalkyl, cycloalkyl, (cycloalkyl)alkyl, (cycloalkyl)alkanoyl, (cycloalkyl)oyl, haloalkanoyl, haloalkyl, heterocycle, (heterocycle)alkanoyl, (heterocycle)oyl, hydroxyalkyl, a nitrogen protecting group, and —C(O)NR₉R₁₀; or R₆ and R₇ together are arylalkylidene; or R₆ and R₇, together with the nitrogen atom to which they are attached, form a heterocycle; and R₉ and R₁₀ are independently selected from the group consisting of hydrogen, alkyl and aryl.

Further definitions and examples of substituents for each moiety in Formula XIII are shown in Craig et al. (U.S. Pat. No. 6,887,863) and Craig et al. (US 2002/0002152). Further embodiments and examples of the compounds of Formula XIII are shown in Craig et al. (U.S. Pat. No. 6,887,863) and Craig et al. (US 2002/0002152). Methods of making compounds of Formula XIII are shown in Craig et al. (U.S. Pat. No. 6,887,863) and Craig et al. (US 2002/0002152).

In other embodiments, the compounds are compounds of Formula XIV:

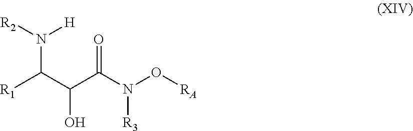

or a pharmaceutically acceptable salt, ester, or prodrug thereof, wherein: R₁ is selected from the group consisting of alkyl, aryl, arylalkyl, cycloalkyl, (cycloalkyl)alkyl, (heterocycle)alkyl, and R₅S-(alkylene)-; wherein each group is drawn with its right-hand end being the end that is attached to the parent molecular moiety; R₃ is selected from the group consisting of hydrogen, alkyl, and arylalkyl; R₅ is selected from the group consisting of alkyl, aryl, arylalkyl, cycloalkyl, and (cycloalkyl)alkyl; and R_{A} is selected from the group consisting of aryl, alkyl, and arylalkyl.

Further definitions and examples of substituents for each moiety in Formula XIV are shown in Craig et al. (U.S. Pat. No. 6,887,863) and Craig et al. (US 2002/0002152). Further embodiments and examples of the compounds of Formula XIV are shown in Craig et al. (U.S. Pat. No. 6,887,863) and Craig et al. (US 2002/0002152). Methods of making compounds of Formula XIV are shown in Craig et al. (U.S. Pat. No. 6,887,863) and Craig et al. (US 2002/0002152).

In other embodiments, the compounds are compounds of Formula XV:

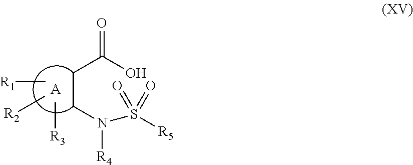

or a pharmaceutically acceptable salt, ester, or prodrug thereof, wherein: A is a five- or six-membered aromatic or non-aromatic ring containing from zero to three atoms selected from the group consisting of nitrogen, oxygen, and sulfur; wherein the five- or six-membered ring is optionally fused to a second five-, six-, or seven-membered aromatic or non-aromatic ring containing from zero to three atoms selected from the group consisting of nitrogen, oxygen, and sulfur; R₁, R₂, and R₃ are independently selected from the group consisting of hydrogen, alkoxy, alkoxyalkyl, alkylsulfanyl, alkylsulfanylalkyl, amino, aminoalkyl, cycloalkyl, (cycloalkyl)alkyl, halo, haloalkoxy, haloalkyl, and hydroxyalkyl. In some embodiments, when A is phenyl, at least one of R₁, R₂ and R₃ is other than hydrogen or C₁ alkyl; R₄ is selected from the group consisting of hydrogen, alkyl, alkylsulfanylalkyl, aryl, and arylalkyl; and R₅ is selected from the group consisting of alkyl, amino, aminoalkyl, aryl, arylalkenyl, arylalkyl, haloalkyl, heteroaryl, heteroarylalkenyl, heteroarylalkyl, and heterocycle.

Further definitions and examples of substituents for each moiety in Formula XV are shown in Comess et al. (US 2004/0068012). Further embodiments and examples of the compounds of Formula XV are shown in Comess et al. (US 2004/0068012). Methods of making compounds of Formula XV are shown in Comess et al. (US 2004/0068012).

In other embodiments, the compounds are compounds of Formula XVI:

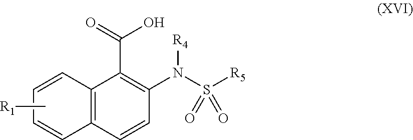

or a pharmaceutically acceptable salt, ester, or prodrug thereof wherein: R₁ is selected from the group consisting of hydrogen, alkoxy, alkoxyalkyl, alkyl, alkylsulfanyl, alkylsulfanylalkyl, amino, aminoalkyl, cycloalkyl, (cycloalkyl)alkyl, halo, haloalkoxy, haloalkyl, and hydroxyalkyl. In some embodiments, when A is phenyl, at least one of R₁, R₂, and R₃ is other than hydrogen or C₁ alkyl; R₄ is selected from the group consisting of hydrogen, alkyl, alkylsulfanylalkyl, aryl, and arylalkyl; and R₅ is selected from the group consisting of alkyl, amino, aminoalkyl, aryl, arylalkenyl, arylalkyl, haloalkyl, heteroaryl, heteroarylalkenyl, heteroarylalkyl, and heterocycle.

Further definitions and examples of substituents for each moiety in Formula XVI are shown in Comess et al. (US 2004/0068012). Further embodiments and examples of the compounds of Formula XVI are shown in Comess et al. (US 2004/0068012). Methods of making compounds of Formula XVI are shown in Comess et al. (US 2004/0068012).

In other embodiments, the compounds are compounds of Formula XVII:

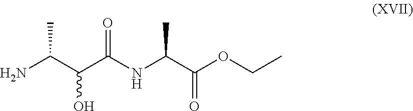

or a pharmaceutically acceptable salt, ester, or prodrug thereof, wherein: R₁ is selected from the group consisting of: CH₂CH₂SCH₃, CH₂CH₂SCH₂CH₃, CH₂CH₂SCH(CH₃)₂, CH₂CH₂CH₂CH₃, C₆H₁₁, CH₂C₆H₁₁, CH₂CH₂C₆H₅, CH₂CH₂C₆H₁₁, CH₂SC(CH₃)₃, CH₂SCH(CH₃)₂, CH₂SCH₂CH₂CH₃, CH₂SCH₂CH(CH₃)₂, CH₂SCH₂C₆H₅, and CH₂SCH₂C₆H₁₁.

Further definitions and examples of substituents for each moiety in Formula XVII are shown in Sheppard et al. (2004, Bioorganic & Medicinal Chemistry Letters 14:865-868). Further embodiments and examples of the compounds of Formula XVII are shown in Sheppard et al. (2004, Bioorganic & Medicinal Chemistry Letters 14:865-868). Methods of making compounds of Formula XVII are shown in Sheppard et al. (2004, Bioorganic & Medicinal Chemistry Letters 14:865-868).

In other embodiments, the compounds are compounds of Formula XVIII:

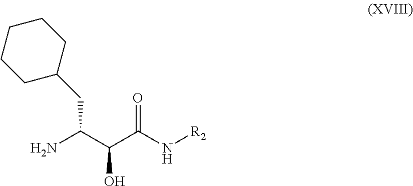

or a pharmaceutically acceptable salt, ester, or prodrug thereof, wherein: R₂ is selected from the group consisting of 1-AlaOEt, d-AlaOEt, HNC(CH₃)₂CO₂Et, 1-AlaOH, 1-LeuOMe, 1-ValOMe, 1-IleOMe, HNCH(CH₃)CH(CH₃)₂, HN(CH₃)₉CH₃, HNPh-3-OCH₃, HNCH₂(2-Naphthyl), HNCH(CH₃)(2-Naphthyl), HN(CH₂)2Ph-2,4-diCl, HNNHC₆H4-4-CH₃, HNOC₆H₅, and HNNHCOC₆H₃-2,5-diCl.

Further definitions and examples of substituents for each moiety in Formula XVIII are shown in Sheppard et al. (2004, Bioorganic & Medicinal Chemistry Letters 14:865-868). Further embodiments and examples of the compounds of Formula XVIII are shown in Sheppard et al. (2004, Bioorganic & Medicinal Chemistry Letters 14:865-868). Methods of making compounds of Formula XVIII are shown in Sheppard et al. (2004, Bioorganic & Medicinal Chemistry Letters 14:865-868).

In other embodiments, the compounds are compounds of Formula XIX:

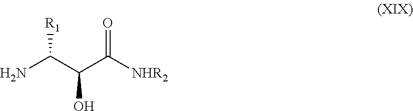

or a pharmaceutically acceptable salt, ester, or prodrug thereof, wherein: R₁ is selected from the group consisting of: CH₃SCH₂CH₂, CH₃CH₂SCH₂CH₂, (CH₂)₂CHCH₂SCH₂, (CH₃)₂CHCH₂SCH₂, and (CH₃)₂CHSCH₂CH₂; and R₂ is selected from the group consisting of: Ethyl (2S)-2-aminopropionate, (S) 1-(1-Naphthyl)ethylamine, and 3-Chloro-benzhydrazide.

Further definitions and examples of substituents for each moiety in Formula XIX are shown in Wang et al. (2003, Cancer Research 63:7861-7869). Further embodiments and examples of the compounds of Formula XIX are shown in Wang et al. (2003, Cancer Research 63:7861-7869). Methods of making compounds of Formula XIX are shown in Wang et al. (2003, Cancer Research 63:7861-7869).

In other embodiments, the compounds are compounds of Formula XX:

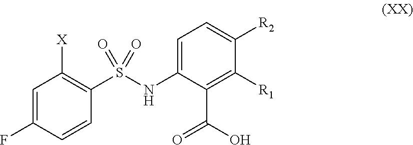

or a pharmaceutically acceptable salt, ester, or prodrug thereof, wherein: X is selected from the group consisting of: H, and Br; R₁ is selected from the group consisting of: H, alkyl, halo, and alkoxy; R₂ is selected from the group consisting of: alkyl, alkenyl, COH, C(OH)CH₂, a five membered saturated or unsaturated heterocyclic ring, wherein the heterocycle is O or N,

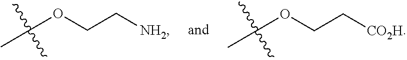

Further definitions and examples of substituents for each moiety in Formula XX are shown in Wang et al. (2007, Bioorganic & Medicinal Chemistry Letters 17:2817-2822). Further embodiments and examples of the compounds of Formula XX are shown in Wang et al. (2007, Bioorganic & Medicinal Chemistry Letters 17:2817-2822). Methods of making compounds of Formula XX are shown in Wang et al. (2007, Bioorganic & Medicinal Chemistry Letters 17:2817-2822).

In other embodiments, the compounds are compounds of Formula XXI:

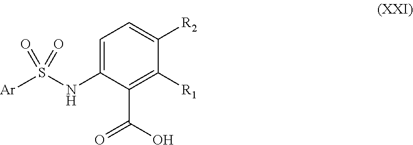

or a pharmaceutically acceptable salt, ester, or prodrug thereof, wherein: R₁ is selected from the group consisting of: alkyl, alkoxy, alkoxyalkyl, and

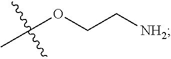

R₂ is selected from the group consisting of: alkyl and a five membered saturated or unsaturated heterocyclic ring, wherein the heterocycle is O and Ar is selected from the group consisting of:

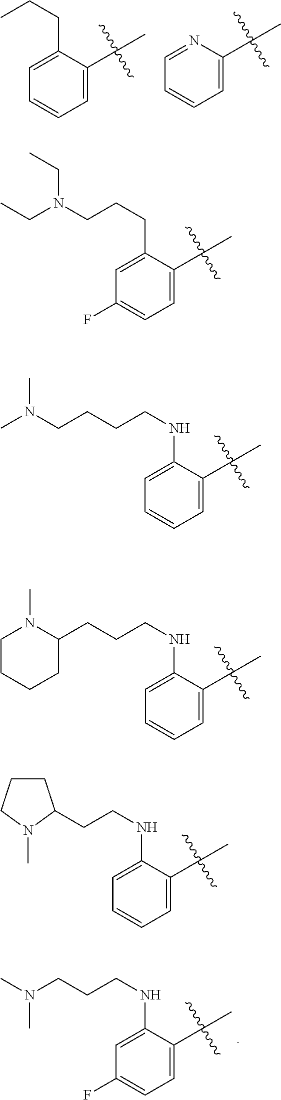

Further definitions and examples of substituents for each moiety in Formula XXI are shown in Wang et al. (2007, Bioorganic & Medicinal Chemistry Letters 17:2817-2822). Further embodiments and examples of the compounds of Formula XXI are shown in Wang et al. (2007, Bioorganic & Medicinal Chemistry Letters 17:2817-2822). Methods of making compounds of Formula XXI are shown in Wang et al. (2007, Bioorganic & Medicinal Chemistry Letters 17:2817-2822).

In other embodiments, the pharmaceutical composition includes a compound selected from the group consisting of:

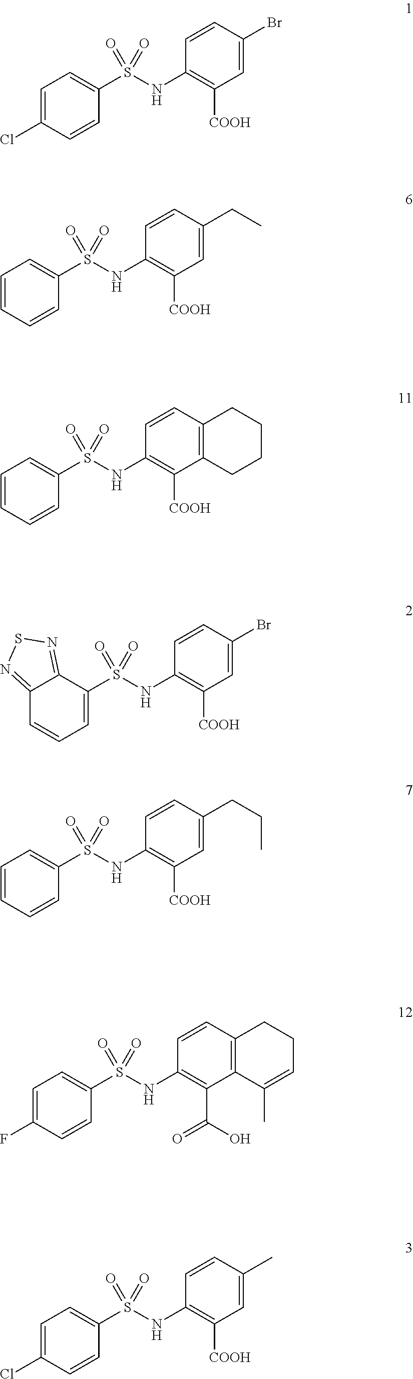

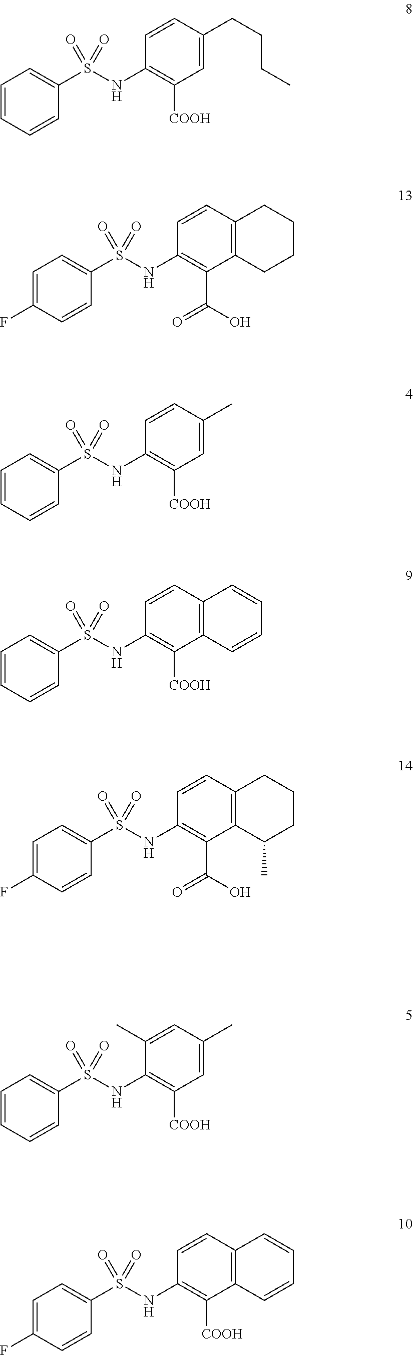

Further definitions and examples of substituents for each moiety in these compounds are shown in Kawai et al. (2006, Bioorganic & Medicinal Chemistry Letters 16:3574-3577). Further embodiments and examples of these compounds are shown in Kawai et al. (2006, Bioorganic & Medicinal Chemistry Letters 16:3574-3577). Methods of making these compounds are shown in Kawai et al. (2006, Bioorganic Medicinal Chemistry Letters 16:3574-3577).

In other embodiments, the pharmaceutical composition includes a conjugated kringle peptide fragment consisting of a functionalized kringle peptide fragment chemically coupled to a functionalized polymer. Further examples of kringle peptide fragments, functionalized polymers, methods of making kringle peptide fragments, methods of making a functionalized polymer, and methods of chemically coupling kringle peptide fragments and functionalized polymers are shown in Henkin et al. (WO 2002/026782),

### Methods

A method of treating obesity in a subject in need thereof is provided herein, comprising non-parenterally administering a therapeutically effective amount of a disclosed compound to said subject. In some embodiments, a contemplated therapeutically effective amount of a disclosed compound as described below, does not substantially modulate or suppress angiogenesis, but is still effective as MetAP-2 inhibitor. The term “angiogenesis” is known to persons skilled in the art, and refers to the process of new blood vessel formation, and is essential for the exponential growth of solid tumors and tumor metastasis. For example, provided herein is a method of treating obesity in a subject in need thereof, comprising administering a therapeutically effective amount of a MetAP-2 inhibitor, e.g., a disclosed compound to said subject, wherein substantially no loss of new blood vessels in fat deposits or other tissue compartments occur as compared to a subject being treated for obesity using an energy restricted diet alone.

Treated subjects used the disclosed methods may have a lower systemic exposure to said MetAP-2 inhibitor as compared to a subject parenterally administered the same of amount of the MetAP-2 inhibitor. In an exemplary embodiment, the disclosed methods may result in less accumulation in the reproductive tract (e.g. testis) of a subject, for example, as compared to the same amount of MetAP-2 inhibitor subcutaneously administered.

Disclosed methods of treating obesity e.g by non-parenterally administering a disclosed compound, may result in decreased body fat and a substantial maintenance of muscle mass in said subject. In certain embodiments, upon administration, fat oxidation is enhanced in a subject as compared to a subject on a restricted food intake diet alone. For example, provided herein is a method of decreasing body fat in an overweight or obese subject in need thereof, comprising administering a therapeutically effective amount of a disclosed compound to said subject resulting in body fat reduction, and wherein said subject substantially maintains muscle mass during the body fat reduction. Such a subject may retain substantially more muscle mass as compared to body fat reduction in a subject using an energy restricted diet alone.

In some embodiments, disclosed methods, upon administration of a disclosed compound e.g. daily or weekly, for about 3, 4, 5 or 6 months or more may result in at least a 5%, 10%, 20%, or 30%, or more weight loss based on the subject's original weight. In an embodiment, weight loss following treatment with therapeutically effective doses of a disclosed compound may substantially cease once a subject attains a normal body composition. Without being limited to an theory, this may be due to reliance of the mechanism on re-establishing tone of adrenergic signal transduction in tissues such as fat, liver, and/or skeletal muscle.

In an embodiment, provided herein is a method of maintaining a specified weight in a formerly obese subject, comprising administering a therapeutically effective amount of a a disclosed compound to said subject.

Also provided herein is a method for controlling or preventing hepatic steatosis in an obese subject being treated for obesity, comprising administering a therapeutically effective amount of a disclosed compound to said subject. In another embodiment, a method for improving liver function in an obese subject is provided, comprising administering a therapeutically effective amount of a disclosed compound to said subject. For example, a method of restoring normal metabolic action in an obese subject in need thereof is provided, comprising administering a therapeutically effective amount of a disclosed compound to said subject. In an embodiment, a method of reducing weight of a subject in a subject in need thereof is provided comprising administering a therapeutically effective amount of a disclosed compound to said subject wherein the metabolic rate of the subject is not substantially reduced as compared to the metabolic rate of a diet only subject on an energy restricted diet alone. In a different embodiment, a method of restoring and/or maintaining thyroid hormone concentrations in an obese subject is provided, comprising administering a therapeutically effective amount of a disclosed compound to said subject.

In an embodiment, a method of improving exercise capacity in a subject in need thereof is provided that comprises administering a therapeutically effective amount of a a disclosed compound to said subject.

Also provided herein is a method of activating brown fat function in a subject in need thereof, comprising administering a therapeutically effective amount of a disclosed compound to said subject.

Contemplated herein is a method of reducing the amount or frequency of administering supplemental insulin in a subject suffering from type 2 diabetes, comprising administering a therapeutically effective amount of a disclosed compound to said subject. Such treatment may be directed to an obese or non-obese subject.

In an embodiment, a method for improving surgical outcome in an obese subject in need thereof by reducing weight of said subject is provided comprising administering a therapeutically effective amount of a disclosed compound to said subject before non-acute surgery, thereby reducing liver and/or abdominal fat in said subject and improving surgical outcome. Such surgeries may include bariatric surgery, cardiovascular surgery, abdominal surgery, or orthopedic surgery.

In addition to being overweight or obese, a subject can further have an overweight- or obesity-related co-morbidities, i.e., diseases and other adverse health conditions associated with, exacerbated by, or precipitated by being overweight or obese. Because being overweight or obese is associated with other adverse health conditions or co-morbidities, for example diabetes, administering a disclosed compound brings a benefit in ameliorating, arresting development of or, in some cases, even eliminating, these overweight- or obesity-related conditions or co-morbidities. In some embodiments, methods provided herein may further include administering at least one other agent that is directed to treatment of these overweight- or obesity-related conditions.

Contemplated other agents include those administered to treat type 2 diabetes such as sulfonylureas (e.g., chlorpropamide, glipizide, glyburide, glimepiride); meglitinides (e.g., repaglinide and nateglinide); biguanides (e.g., metformin); thiazolidinediones (rosiglitazone, troglitazone, and pioglitazone); glucagon-like 1 peptide mimetics (e.g. exenatide and liraglutide); sodium-glucose cotransporter inhibitors (e.g., dapaglillozin), renin inhibitors, and alpha-glucosidase inhibitors (e.g., acarbose and meglitol), and/or those administered to treat cardiac disorders and conditions, such hypertension, dyslipidemia, ischemic heart disease, cardiomyopathy, cardiac infarction, stroke, venous thromboembolic disease and pulmonary hypertension, which have been linked to overweight or obesity, for example, chlorthalidone; hydrochlorothiazide; indapamide, metolazone; loop diuretics (e.g., bumetanide, ethacrynic acid, furosemide, lasix, torsemide); potassium-sparing agents (e.g., amiloride hydrochloride, spironolactone, and triamterene); peripheral agents (e.g., reserpine); central alpha-agonists (e.g., clonidine hydrochloride, guanabenz acetate, guanfacine hydrochloride, and methyldopa); alpha-blockers (e.g., doxazosin mesylate, prazosin hydrochloride, and terazosin hydrochloride); beta-blockers acebutolol, atenolol, betaxolol, nisoprolol fumarate, carteolol hydrochloride, metoprolol tartrate, metoprolol succinate, Nadolol, penbutolol sulfate, pindolol, propranolol hydrochloride, and timolol maleate); combined alpha- and beta-blockers carvedilol and labetalol hydrochloride); direct vasodilators (e.g., hydralazine hydrochloride and minoxidil); calcium antagonists (e.g., diltiazem hydrochloride and verapamil hydrochloride); dihydropyridines (e.g., amlodipine besylate, felodipine, isradipine, nicardipine, nifedipine, and nisoldipine); ACE inhibitors (benazepril hydrochloride, captopril, enalapril maleate, fosinopril sodium, lisinopril, moexipril, quinapril hydrochloride, ramipril, trandolapril); angiotensin II receptor blockers (e.g., losartan potassium, valsartan, and Irbesartan); and combinations thereof, as well as statins such as mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin, typically for treatment of dyslipidemia.

Other agents that may be co-administered (e.g. sequentially or simultaneously) include agents administered to treat ischemic heart disease including statins, nitrates (e.g., Isosorbide Dinitrate and isosorbide Mononitrate), beta-blockers, and calcium channel antagonists, agents administered to treat cardiomyopathy including inotropic agents (e.g., Digoxin), diuretics (e.g., Furosemide), ACE inhibitors, calcium antagonists, anti-arrhythmic agents Sotolol, Amiodarone and Disopyramide), and beta-blockers, agents administered to treat cardiac infarction including ACE inhibitors, Angiotensin II receptor blockers, direct vasodilators, beta blockers, anti-arrhythmic agents and thrombolytic agents (e.g., Alteplase, Retaplase, Tenecteplase, Anistreplase, and Urokinase), agents administered to treat strokes including anti-platelet agents (e.g., Aspirin, Clopidogrel, Dipyridamole, and Ticlopidine), anticoagulant agents (e.g. Heparin), and thrombolytic agents, agents administered to treat venous thromboembolic disease including anti-platelet agents, anticoagulant agents, and thrombolytic agents, agents administered to treat pulmonary hypertension include inotropic agents, anticoagulant agents, diuretics, potassium (e.g., K-dur), vasodilators (e.g., Nifedipine and Diltiazem), Bosentan, Epoprostenol, and Sildenafil, agents administered to treat asthma include bronchodilators, anti-inflammatory agents, leukotriene blockers, and anti-Ige agents. Particular asthma agents include Zatirlukast, Flunisolide, Triamcinolone, Beclomethasone, Terbutaline, Fluticasone, Formoterol, Beclomethasone, Salmeterol, Theophylline, and Xopenex, agents administered to treat sleep apnea include Modafinil and amphetamines, agents administered to treat nonalcoholic fatty liver disease include antioxidants (e.g., Vitamins E and C), insulin sensitizers (Metformin, Pioglitazone, Rosiglitazone, and Betaine), hepatoprotectants, and lipid-lowering agents, agents administered to treat osteoarthritis of weight-bearing joints include Acetaminophen, non-steroidal anti-inflammatory agents (e.g., Ibuprofen, Etodolac, Oxaprozin, Naproxen, Diclofenac, and Nabumetone), COX-2 inhibitors (e.g., Celecoxib), steroids, supplements (e.g. glucosamine and chondroitin sulfate), and artificial joint fluid, agents administered to treat Prader-Willi Syndrome include human growth hormone (HGH), somatropin, and weight loss agents (e.g., Orlistat, Sibutramine, Methamphetamine, Ionamin, Phentermine, Bupropion, Diethylpropion, Phendimetrazine, Benzphetermine, and Topamax), agents administered to treat polycystic ovary syndrome include insulin-sensitizers, combinations of synthetic estrogen and progesterone, Spironolactone, Eflornithine, and Clomiphene, agents administered to treat erectile dysfunction include phosphodiesterase inhibitors (e.g., Tadalafil, Sildenafil citrate, and Vardenafil), prostaglandin E analogs (e.g., Alprostadil), alkaloids (e.g., Yohimbine), and testosterone, agents administered to treat infertility include Clomiphene, Clomiphene citrate, Bromocriptine, Gonadotropin-releasing Hormone (GnRH), GnRH agonist, GnRH antagonist, Tamoxifen/nolvadex, gonadotropins, Human Chorionic Gonadotropin (HCG), Human Menopausal Gonadotropin (HmG), progesterone, recombinant follicle stimulating hormone (FSH), Urofollitropin, Heparin, Follitropin alfa, and Follitropin beta, agents administered to treat obstetric complications include Bupivacaine hydrochloride, Dinoprostone PGE2, Meperidine HCl, Ferro-folic-500/iberet-folic-500, Meperidine, Methylergonovine maleate, Ropivacaine HCl, Nalbuphine HCl, Oxymorphone HCl, Oxytocin, Dinoprostone, Ritodrine, Scopolamine hydrobromide, Sufentanil citrate, and Oxytocic, agents administered to treat depression include serotonin reuptake inhibitors (e.g., Fluoxetine, Escitalopram, Citalopram, Paroxetine, Sertraline, and Venlafaxine); tricyclic antidepressants (e.g., Amitriptyline, Amoxapine, Clomipramine, Desipramine, Dosulepin hydrochloride, Doxepin, lmipramine, Iprindole, Lofepramine, Nortriptyline, Opipramol, Protriptyline, and Trimipramine); monoamine oxidase inhibitors (e.g., Isocarboxazid, Moclobemide, Phenelzine, Tranylcypromine, Selegiline, Rasagiline, Nialamide, Iproniazid, Iproclozide, Toloxatone, Linezolid, Dienolide kavapyrone desmethoxyyangonin, and Dextroamphetamine); psychostimulants (e.g., Amphetamine, Methamphetamine, Methylphenidate, and Arecoline); antipsychotics (e.g., Butyrophenones, Phenothiazines, Thioxanthenes, Clozapine, Olanzapine, Risperidone, Quetiapine, Ziprasidone, Amisulpride, Paliperidone, Symbyax, Tetrabenazine, and Cannabidiol); and mood stabilizers (e.g., Lithium carbonate, Valproic acid, Divalproex sodium, Sodium valproate, Lamotrigine, Carbamazepine, Gabapentin, Oxcarbazepine, and Topiramate), agents administered to treat anxiety include serotonin reuptake inhibitors, mood stabilizers, benzodiazepines (e.g., Alprazolam, Clonazepam, Diazepam, and Lorazepam), tricyclic antidepressants, monoamine oxidase inhibitors, and beta-blockers, and other weight loss agents, including serotonin and noradrenergic re-uptake inhibitors; noradrenergic re-uptake inhibitors; selective serotonin re-uptake inhibitors; and intestinal lipase inhibitors. Particular weight loss agents include orlistat, sibutramine, methamphetamine, ionamin, phentermine, bupropion, diethylpropion, phendimetrazine, benzphetermine, and topamax.

In some embodiments, contemplated methods may further comprising assessing one or more indices of on-going weight loss, e.g. the ketone body production level in a subject; and optionally adjusting the amount administered; thereby optimizing the therapeutic efficacy of the disclosed compound.

### Administration and Formulation

Pharmaceutical compositions having compounds disclosed herein can be administered in the form of a free acid. Alternatively, a salt can be prepared by reacting compounds disclosed herein with a suitable base. Pharmaceutically acceptable salts illustratively include those that can be made using the following bases: ammonia, L-arginine, benethamine, benzathene, betaine, bismuth, calcium hydroxide, choline, deanol, diethanolamine, diethylamine, 2-(diethylamino)ethanol, ethylenediamine, N-methylglucamine, hydrabamine, 1H-imidazole, lysine, magnesium hydroxide, 4-(2-hydroxyethyl)morpholine, piperazine, potassium hydroxide, 1-(2-hydroxyethyl)pyrrolidine, sodium hydroxide, triethanolamine, zinc hydroxide, diclyclohexlamine, or any other electron pair donor (as described in Handbook of Pharmaceutical Salts, Stan & Wermuth, VHCA and Wiley, Uchsenfurt-Hohestadt Germany, 2002). Esters disclosed herein may be prepared by reacting compounds disclosed herein with the appropriate acid under standard esterification conditions described in the literature (Houben-Weyl 4th Ed. 1952, Methods of Organic Synthesis). Suitable esters include ethyl methanoate, ethyl ethanoate, ethyl propanoate, propyl methanoate, propyl ethanoate, and methyl butanoate.

Compounds disclosed herein may be administered using any amount and any route of administration effective for treating a subject having an overweight or obese condition without substantially reducing lean body mass of the subject. Thus, the expression “amount effective for treating a subject having an overweight or obese condition”, as used herein, refers to a pharmaceutical composition having a sufficient amount of compounds disclosed herein, or salts or esters thereof, to beneficially result in weight loss without deleterious side effects, such as substantial reduction of lean body mass of the subject.

Dosage and administration are adjusted to provide sufficient levels of compounds disclosed herein, or salts or esters thereof to maintain the desired effect. Additional factors that may be taken into account include the severity of the disease state, e.g., overweight, obese, or morbidly obese; age, and gender of the subject; diet, time and frequency of administration; route of administration; drug combinations; reaction sensitivities, and tolerance/response to therapy. Long acting pharmaceutical compositions might be administered hourly, twice hourly, every three to four hours, daily, twice daily, every three to four days, every week, or once every two weeks depending on half-life and clearance rate of the particular composition.

Therapeutic efficacy and toxicity of compounds disclosed herein, or salts or esters thereof, can be determined by standard pharmaceutical procedures. For example, therapeutic efficacy and toxicity can be determined by minimal efficacious dose or NOAEL (no observable adverse effect level). Alternatively, an ED50 (the dose is therapeutically effective in 50% of the population) and LD50 (the dose is lethal to 50% of the population) can be determined in cell cultures or experimental animals. The dose ratio of toxic to therapeutic effects is the therapeutic index, and it can be expressed as the ratio, LD50/ED50. Pharmaceutical compositions that exhibit large therapeutic indices are preferred.

Compounds disclosed herein, or salts or esters thereof, may be formulated in dosage unit form for ease of administration and uniformity of dosage. In general, the total daily usage of the compositions disclosed herein will be decided by the attending physician within the scope of sound medical judgment. The therapeutically effective dose can be estimated initially either in cell culture assays or in animal models, as provided herein, usually mice, but also potentially from rats, rabbits, dogs, or pigs. The animal model provided herein is also used to achieve a desirable concentration and total dosing range and route of administration. Such information can then be used to determine useful doses and routes for administration in humans.

Contemplated herein are formulations suitable for non-parenteral administration of a disclosed compound. For example, in certain embodiments, a subject may have a lower systemic exposure (e.g. at least about 2, 3, 5, 10, 20, or at least about 30% less systemic exposure) to the non-parenterally administered (e.g. oral administration) of a disclosed compound as compared to a subject parenterally administered (e.g. subcutaneously) the same dose of the disclosed compound.

Contemplated non-parenteral administration includes oral, buccal, transdermal (e.g. by a dermal patch), topical, inhalation, or sublingual administration, or e.g., ocular, pulmonary, nasal, rectal or vaginal administration.

In another embodiment, provided herein are effective dosages, e.g. a daily dosage of a disclosed compound, that may not substantially modulate or suppress angiogenesis. For example, provided here are methods that include administering doses of a disclosed compound that are effective for weight loss, but are significantly smaller doses than that necessary to modulate and/or suppress angiogenesis (which may typically require about 12.5 mg/kg to about 50 mg/kg or more). For example, contemplated dosage of a disclosed compound in the methods described herein may include administering about 25 mg/day, about 10 mg/day, about 5 mg/day, about 3 mg/day, about 2 mg/day, about 1 mg/day, about 0.75 mg/day, about 0.5 mg/day, about 0.1 mg/day, about 0.05 mg/day, or about 0.01 mg/day. For example, an effective amount of the drug for weight loss in a subject may be about 0.0001 mg/kg to about 25 mg/kg of body weight per day. For example, a contemplated dosage may from about 0.001 to 10 mg/kg of body weight per day, about 0.001 mg/kg to 1 mg/kg of body weight per day, about 0.001 mg/kg to 0.1 mg/kg of body weight per day or about 0.005 to about 0.04 mg/kg or about 0.005 to about 0.049 mg/kg of body weight a day.

Contemplated methods may include administration of a composition comprising a disclosed compound, for example, hourly, twice hourly, every three to four hours, daily, twice daily, 1, 2, 3 or 4 times a week, every three to four days, every week, or once every two weeks depending on half-life and clearance rate of the particular composition or inhibitor.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a weight loss target, is achieved. For example, when about loss of about 20% body weight, about 30% body weight or more has been achieved. A treatment regimen can include a corrective phase, during which a disclosed compound dose sufficient to provide reduction of excess adiposity is administered, followed by a maintenance phase, during which a lower dose sufficient to prevent re-development of excess adiposity is administered.

For pulmonary (e.g., intrabronchial) administration, compounds disclosed herein, or a salt or ester thereof, can be formulated with conventional excipients to prepare an inhalable composition in the form of a fine powder or atomizable liquid.

For ocular administration, compounds disclosed herein, or a salt or ester thereof, can be formulated with conventional excipients in the form of eye drops or an ocular implant. Among excipients useful in eye drops are viscosifying or gelling agents, to minimize loss by lacrimation through improved retention in the eye.

Liquid dosage forms for oral or other systemic administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for topical or transdermal administration of an disclosed pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, cutaneous routes of administration are achieved with aqueous drops, a mist, an emulsion, or a cream.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compositions for rectal or vaginal administration may be suppositories which can be prepared by mixing the active agent(s) disclosed herein with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. Alternatively, formulations suitable for use with the methods disclosed herein are incorporated into chewable tablets, crushable tablets, tablets that dissolve rapidly in within the mouth, or mouthwash. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

In addition to being overweight or obese, a subject can further have an overweight- or obesity-related co-morbidities, i.e., diseases and other adverse health conditions associated with, exacerbated by, or precipitated by being overweight or obese. Thus, a method of treating a subject having an overweight- or obesity-related condition is provided herein, the method involving administering to the subject a therapeutically effective amount of compounds disclosed herein, or a salt or ester thereof, such that the amount administered does not substantially reduce lean body mass of the subject.

For example, Type II diabetes has been associated with obesity. Certain complications of Type II diabetes, e.g., disability and premature death, can be prevented, ameliorated, or eliminated by sustained weight loss (Astrup, A. Pub health Nutr (2001) 4:499-5 15).

Cardiac disorders and conditions, for example hypertension, dyslipidemia, ischemic heart disease, cardiomyopathy, cardiac infarction, stroke, venous thromboembolic disease and pulmonary hypertension, have been linked to overweight or obesity. For example, hypertension has been linked to obesity because excess adipose tissue secretes substances that are acted on by the kidneys, resulting in hypertension. Additionally, with obesity there are generally higher amounts of insulin produced (because of the excess adipose tissue) and this excess insulin also elevates blood pressure. A major treatment option of hypertension is weight loss.

Respiratory disorders and conditions such as obesity-hypoventilation syndrome, asthma, and obstructive sleep apnea, have been linked to being overweight or obese. Elamin (Chest (2004) 125:1972-1974) discusses a link between being overweight or obese and asthma. Kessler et al. (Eur Respir J (1996) 9:787-794) discusses a link between being overweight or obese and obstructive sleep apnea. Hepatic disorders and conditions, such as nonalcoholic fatty liver disease, have been linked to being overweight or obese. Tolman et al. (Ther Clin Risk Manag (2007) 6:1153-1163) discusses a link between being overweight or obese and nonalcoholic fatty liver disease.

Because being overweight or obese is associated with the above conditions, administering pharmaceutical compositions having compounds disclosed herein bring a benefit in ameliorating, arresting development of or, in some cases, even eliminating, these overweight- or obesity-related conditions.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLE

### Example 1

### An Orally Administered Compound Disclosed Herein Causes Weight Loss in Diet-Induced Obese Mice

A weight loss study was conducted in obese mice. The mice in this study were not genetically obese, but prior to and during the study, obesity was induced by a high-fat diet. Twelve week-old C57BL/6NTac mice, maintained on a 60% fat diet prior to and during the study, were separated into two groups, with eight animals per group. Average body weight of the mice was approximately 47 g at the start of the study.

One group was orally administered 1.0 mg/kg of compound H in 10% gelucire in deionised water, and one group was administered 10% gelucire in deionised water (vehicle). Compound H is as follows:

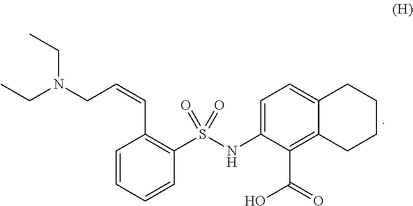

Mice received administrations once a day for 7 days. After 7 days, the average weight loss of mice administered compound H was 13.2%.

What is claimed is:

1. A method of treating a subject for an overweight or obese condition, the method comprising: administering non-parenterally to the subject in need thereof, a therapeutically effective amount of a pharmaceutical composition comprising a compound of Formula III: 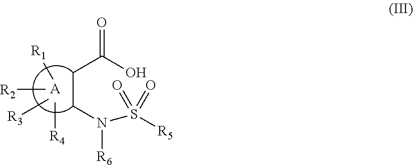
or a pharmaceutically acceptable salt, ester, or prodrug thereof,
wherein:
A is a five- or six-membered aromatic or non-aromatic ring containing from zero to three atoms selected from the group consisting of nitrogen, oxygen, and sulfur; wherein the five- or six-membered ring is optionally fused to a second five-, six-, or seven-membered aromatic or non-aromatic ring containing from zero to three atoms selected from the group consisting of nitrogen, oxygen, and sulfur;
R₁, R₂ and R₃ are independently selected from the group consisting of hydrogen, alkenyl, alkoxy, alkoxyalkyl, alkoxycarbonyl, alkoxycarbonylalkyl, alkyl, alkylcarbonyloxy, alkylidene, alkylsulfanyl, alkylsulfanylalkyl, alkylsulfonyl, alkylsulfonylalkyl, amino, aminoalkyl, aminoalkenyl, aminoalkoxy, aminocarbonylalkenyl, aryl, carboxyalkenyl, carboxyalkyl, cyano, cycloalkyl, (cycloalkyl)alkyl, halo, haloalkoxy, haloalkyl, (heterocycle)alkyl, hydroxy, hydroxyalkyl, nitro;
R₄ is selected from the group consisting of hydrogen, alkenyl, alkoxy, alkoxyalkyl, alkoxycarbonyl, alkyl, alkylcarbonyl, alkylsulfonyl, alkylsulfanyl, alkylsulfanylalkyl, carboxy, cyano, cyanoalkyl, cycloalkyl, (cycloalkyl)alkyl, halo, haloalkoxy, haloalkyl, heteroaryl, heterocycle, heterocyclealkyl, heterocyclealkenyl, hydroxy, hydroxyalkyl, nitro, phenyl, phenylsulfonyl, R_{c4}R_{d4}N—, R_{c4}R_{d4}Nalkyl, R_{c4}R_{d4}Nalkenyl, R_{c4}R_{d4}Nalkynyl, R_{c4}R_{d4}Nalkoxy, R_{c4}R_{d4}Nalkoxycarbonyl, R_{c4}R_{d4}Ncarbonyl, R_{c4}R_{d4}Ncycloalkyl, R_{c4}R_{d4}Nalkylcycloalkyl, R_{c4}R_{d4}N(cycloalkyl)alkyl, R_{c4}R_{d4}Nsulfinyl, Rₑ₄R_{f4}Nalkyl(R_{c4})N—, Rₑ₄R_{f4}Nalkyl(R_{c4})Ncarbonyl, Rₑ₄R_{f4}Nalkyl(R_{c4})Ncarbonylalkenyl, Rₑ₄R_{f4}Nalkylcarbonyl(R₄)N—, Rₑ₄R_{f4}Nalkoxycarbonyl(R_{c4})N—, R_{c4}R_{d4}Nalkylsulfanyl, R_{c4}R_{d4}Nalkylsulfinyl, R_{c4}R_{d4}Nalkylsulfonyl, R_{g4}Rⱼ₄Nalkyl(Rₑ₄)Ncarbonyl(R_{c4})N—; wherein the phenyl group, the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c4} R_{d4}, Rₑ₄, R_{f4}, R_{g4} and Rⱼ₄ are each independently selected from the group consisting of hydrogen, alkoxyalkyl, alkyl, alkylcarbonyl, aminoalkyl, cycloalkyl, (cycloalkyl)alkyl, heterocycle and phenyl, or each individual pair of R_{c4} and R_{d4}, or Rₑ₄ and R_{f4}, or R_{g4} and Rⱼ₄ taken together with the nitrogen atom they are each attached form a heterocycle;
R₅ is selected from the group consisting of alkyl, amino, aminoalkyl, aryl, arylalkenyl, arylalkyl, haloalkyl, heteroaryl, heteroarylalkenyl, heteroarylalkyl, heterocycle, heterocyclealkyl and heterocyclealkenyl, wherein aryl, the aryl group of arylalkenyl, the aryl group of arylalkyl, the heteroaryl, the heteroaryl of heteroarylalkenyl, the heteroaryl of heteroarylalkyl, and the heterocycle of R₅ may be optionally substituted with 1, 2 or 3 substituents independently selected from the group consisting of alkenyl, alkoxy, alkoxycarbonyl, alkyl, alkylcarbonyl, alkylsulfonyl, aminoalkyl, phenyl, phenylsulfonyl, carboxy, cyano, cyanoalkyl, halo, haloalkoxy, haloalkyl, heteroaryl, heterocycle, heterocyclealkyl, heterocyclealkenyl, hydroxy, nitro, R_{c5}R_{d5}N—, R_{c5}R_{d5}Nalkyl, R_{c5}R_{d5}Nalkenyl, R_{c5}R_{d5}Nalkynyl, R_{c5}R_{d5}Nalkoxy, R_{c5}R_{d5}Nalkoxycarbonyl, R_{c5}R_{d5}Ncarbonyl, R_{c5}R_{d5}Ncycloalkyl, R_{c5}R_{d5}Nalkylcycloalkyl, R_{c5}R_{d5}Ncycloalkylalkyl, R_{c5}R_{d5}Nsulfinyl, Rₑ₅R_{f5}Nalkyl(R_{c5})N—, Rₑ₅R_{f5}Nalkyl(R_{c5})Ncarbonyl, Rₑ₅R_{f5}Nalkyl(R_{c5})Ncarbonylalkenyl, Rₑ₅R_{f5}Nalkylcarbonyl(R_{c5})N—, Rₑ₅R_{f5}Nalkoxycarbonyl(R_{c5})N—, R_{c5}R_{d5}Nalkylsulfanyl, R_{c5}R_{d5}Nalkylsulfinyl, R_{c5}R_{d5}Nalkylsulfonyl, R_{g5}Rⱼ₅Nalkyl(Rₑ₅)Ncarbonyl(R_{c5})N—; wherein the phenyl group of phenylsulfonyl, the heteroaryl, the heterocycle, the heterocycle of heterocyclealkyl, the heterocycle of heterocyclealkenyl may be optionally substituted with 1, 2 or 3 substituents selected from the group consisting of alkoxy, alkyl, cyano, halo, haloalkoxy, haloalkyl, and nitro; and wherein R_{c5}, R_{d5}, Rₑ₅, R_{f5}, R_{g5} and Rⱼ₅ are each independently selected from the group consisting of hydrogen, alkoxyalkyl, alkyl, alkylcarbonyl, aminoalkyl, cycloalkyl, (cycloalkyl)alkyl, heterocycle and phenyl;
R₆ is selected from the group consisting of hydrogen, alkyl, alkylsulfanylalkyl, aryl, and arylalkyl.

2. The method of claim 1, wherein the compound is of Formula IX: 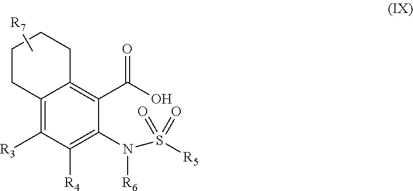
wherein: R₃, R₄, R₅, and R₆ are as described, and R₇ is selected from the group consisting of hydrogen, C₁-C₃ alkyl, C2-C₃ alkenyl, C₂-C₃ alkoxy, halo, haloalkyl, haloalkoxy, RₐR_{b}N— and RₐR_{b}Nalkoxy, wherein Rₐ and R_{b} are each independently selected from the group consisting of hydrogen and alkyl.

3. The method of claim 2, wherein R₅ is an optionally substituted phenyl.

4. A method of treating a subject for an overweight or obese condition, the method comprising: administering non-parenterally to the subject in need thereof, a therapeutically effective amount of a pharmaceutical composition comprising a compound

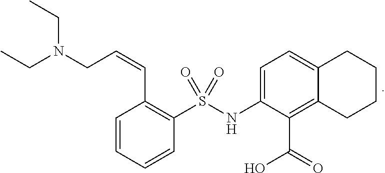

5. The method of claim 1, wherein the subject has a Body Mass Index measurement selected from the group consisting of: at least about 25 kg/m², at least about 30 kg/m², and at least about 40 kg/m².

6. The method of claim 1, wherein the pharmaceutical composition is administered orally, buccally, sublingually, transdermally, via inhalation, or rectally.

7. The method of claim 1, wherein administration results in decreased body fat and a substantial maintenance of muscle mass in the subject.

8. The method of claim 1, wherein upon administration, fat oxidation is enhanced as compared to a subject on a restricted food intake diet alone.

9. The method of claim 1, wherein substantially no loss of new blood vessels in fat deposits occur as compared to a subject being treated for obesity using an energy restricted diet alone.

10.-17. (canceled)

18. A method of restoring and/or maintaining thyroid hormone concentrations in an obese subject, comprising administering a therapeutically effective amount of a pharmaceutical composition comprising a compound of Formula III to said subject.

19. The method of claim 1, wherein said therapeutically effective amount does not substantially modulate or suppress angiogenesis.

20. The method of claim 1, wherein said subject has a lower systemic exposure to said compound as compared to a subject parenterally administered the same amount of the compound.
